(19) United States
(12) Patent Application Publication
(10) Pub. No.: US 2020/0205670 A1
(43) Pub. Date: Jul. 2, 2020

(54) METHODS AND APPARATUSES FOR PHOTOACOUSTIC IMAGING

(71) Applicant: Butterfly Network, Inc., Guilford, CT (US)

(72) Inventors: Tyler S. Ralston, Clinton, CT (US); Murad Omar, New Haven, CT (US); Lawrence C. West, San Jose, CT (US); Jonathan M. Rothberg, Guilford, CT (US)

(73) Assignee: Butterfly Network, Inc., Guilford, CT (US)

(21) Appl. No.: 16/728,844

(22) Filed: Dec. 27, 2019

Related U.S. Application Data
(60) Provisional application No. 62/785,962, filed on Dec. 28, 2018.

(51) Int. Cl. A61B 5/00

(52) U.S. Cl. CPC A61B 5/0095; B06B 1/0607

(57) ABSTRACT

Methods and apparatuses are provided for photoacoustic imaging. One such apparatus may include an ultrasound-on-a-chip device attached to a housing, an optical emitter attached to the housing, and a controller enclosed at least partially in the housing. The ultrasound-on-a-chip device may include a plurality of ultrasonic transducers. The optical emitter may include an array of diodes arranged at a periphery of the plurality of ultrasonic transducers. The controller may be configured to control the optical emitter to emit pulses of light, to control the plurality of ultrasonic transducers to detect ultrasonic waves emitted from a target to be imaged in response to exposure to the pulses of light, and to convert the ultrasonic waves to digital signals. For example, the optical emitter may be controlled to emit chirped optical pulses. The digital signals may be processed by the controller to produce image-formation data.

### CROSS-REFERENCE TO RELATED APPLICATION(S)

This application claims the benefit under 35 U.S.C. § 119(e) of U.S. Provisional Application Ser. No. 62/785,962, entitled “METHODS AND APPARATUSES FOR PHOTOACOUSTIC IMAGING” filed on Dec. 28, 2018, under Attorney Docket Number B1348.70125US00, which is incorporated herein by reference in its entirety.

### FIELD

Generally, aspects of the technology described herein relate to ultrasound systems that detect ultrasonic waves produced when a target is exposed to light irradiation, and that produce an image of the target from the ultrasonic waves.

### BACKGROUND

Conventional systems for photoacoustic imaging generally include an ultrasound probe arranged relative to a fiber-optic device that delivers light from a high-power laser (e.g., a Q-switched Nd:YAG laser, a dye laser, and the like) to a target. Such systems tend to be large, complex, and expensive, and typically are located in large medical facilities (e.g., a hospital) and are operated by medical professionals that are trained in both ultrasound technology and laser technology. The specialized training of medical professionals to operate photoacoustic imaging systems may take years and add to the costs of maintaining and operating these systems.

### SUMMARY

The inventors have appreciated the need for a photoacoustic imaging system that is compact and portable, that can be produced at a cost significantly lower than that of a conventional photoacoustic imaging system, and that can readily allow multiple wavelengths to be used sequentially or simultaneously without requiring retooling or reconfiguration of the system. To this end, disclosed herein is technology for a hand-held photoacoustic imaging device that may be battery-powered, portable, and sized to be held in an operator's hand. The probe may include low-cost diodes for illuminating or irradiating a target with light to produce the photoacoustic effect, and ultrasonic transducers for detecting ultrasonic waves emitted from the target.

According to aspects of the present technology, a photoacoustic imaging apparatus is provided. That apparatus may include an ultrasound-on-a chip device attached to a housing, the ultrasound-on-a-chip device comprising a plurality of transducers; an optical emitter attached to the housing; and a controller at least partially disposed in the housing. The optical emitter may comprise an array of diodes arranged at a periphery of the plurality of ultrasonic transducers. The controller may be configured to control the optical emitter to emit pulses of light, to control the plurality of ultrasonic transducers to detect and measure ultrasonic waves emitted from a target to be imaged in response to exposure to the pulses of light, and to convert the ultrasonic waves to a digital signal.

In an aspect of the present technology, the ultrasound-on-a-chip device may comprise the controller. The controller may be configured to process the digital signal to produce image-formation data.

In another aspect of the present technology, the array of diodes may comprise a plurality of light-emitting diodes (LEDs), or a plurality of laser diodes, or a combination of at least one LED and at least one laser diode.

In some variations of this aspect, the array of diodes may comprise the plurality of LEDs, which may include at least two LEDs that emit light having different wavelengths from each other. In some variations of this aspect, the array of diodes may comprise the plurality of laser diodes, which may include at least two laser diodes that emit light having different wavelengths from each other. The controller may be configured to control the optical emitter to perform tunable imaging of the target using the different wavelengths. For example, the controller may be configured to control the tunable imaging of the target by controlling the optical emitter to emit light sequentially according to the different wavelengths. In another example, the controller may be configured to control the optical emitter to emit light selectively according to a desired wavelength for imaging the target.

In another aspect of the present technology, the optical emitter may comprises at least one Q-switched laser, which may include at least one diode-pumped solid-state (DPSS) laser.

In a further aspect of the present technology, the array of diodes may be arranged to surround a periphery of the plurality of ultrasonic transducers. The periphery of the plurality of ultrasonic transducers may be shaped as a square or a rectangle, or may be shaped as a circle or an oval, or may have any other suitable shape.

In another aspect of the present technology, the photoacoustic imaging apparatus may further comprise a handle sized to be held by a single hand of an operator. The handle may form at least a portion of the housing.

In yet another aspect of the present technology, the photoacoustic imaging apparatus may further comprise a battery disposed in the housing. The battery may be configured to provide electric power to the ultrasonic transducers, the optical emitter, and the controller.

In a further aspect of the present technology, the plurality of ultrasonic transducers may comprise microelectromechanical system (MEMS) transducers. For example, the MEMS transducers may comprise any one or any combination of: at least one capacitive micromachined ultrasonic transducer (CMUT), at least one CMOS (complementary metal-oxide-semiconductor) ultrasonic transducer (CUT), and at least one piezoelectric micromachined ultrasonic transducer (PMUT). Optionally, in addition to the MEMS transducers, the plurality of ultrasonic transducers may further comprise at least one piezoelectric transducer that is not a MEMS transducer.

In another aspect of the present technology, the plurality of ultrasonic transducers may operate in a broadband-frequency range of approximately 1 MHz to approximately 10 MHz.

In a variation of this aspect, the plurality of ultrasonic transducers may be comprised of at least one multi-function chip configured to perform a linear scan of the target, a curvilinear scan of the target, a sector scan of the target, and a 2D or 3D scan of the target. In another variation of this aspect, the plurality of ultrasonic transducers may be controlled by the controller to perform an electronic scan of the target.

In a further aspect of the present technology, the plurality of ultrasonic transducers may operate in a high-frequency range of approximately 5 MHz to approximately 40 MHz.

In a variation of this aspect, the plurality of ultrasonic transducers may be arranged on at least one chip (e.g., at least one ultrasound-on-a-chip device) comprising a plurality of elements arrayed at a pitch in a range of approximately 40 μm to approximately 300 μm. For example, the range may be approximately 140 μm to approximately 160 μm. The spacing between elements may be less than the wavelength (e.g., a half-wavelength spacing for a bistatic array of elements, a quarter-wavelength spacing for a monostatic array of elements). In another variation of this aspect, each chip of the at least one chip may have approximate dimensions of 50 mm×10 mm. In a further variation, each chip may have approximate dimensions of 30 mm×20 mm.

In another aspect of the present technology, the plurality of ultrasonic transducers may operate in an ultra-high-frequency range of approximately 40 MHz to approximately 180 MHz.

In yet another aspect of the present technology, the plurality of ultrasonic transducers may comprise a plurality of MEMS transducers configured to detect ultrasonic waves emitted from a volume that extends below a surface of the target. The controller may be configured to process the ultrasonic waves to produce a 2D or 3D image of the target.

In a further aspect of the present technology, the controller may be configured to control the array of diodes to perform pulsed emission at approximately 10 Hz at an average power of approximately 0.2 Watts/cm². In a variation of this aspect, the controller may be configured to process pulse trains of approximately 10 seconds or longer. For example, each of the pulse trains may have a duration in a range of approximately 10 seconds to approximately 30 seconds.

In another aspect of the present technology, the controller may be configured to perform chirp processing on pulse trains and to control the array of diodes to perform emission of chirped pulses.

According to aspects of the present technology, a photoacoustic imaging system is provided. The system may comprise a photoacoustic apparatus, a controller, and a signal processor. The apparatus may include a plurality of ultrasonic transducers attached to a housing, and an optical emitter attached to the housing. The optical emitter may comprise an array of diodes arranged at a periphery of the plurality of ultrasonic transducers. The controller may be configured to control the optical emitter to emit pulses of light, and to control the plurality of ultrasonic transducers to detect and measure ultrasonic waves emitted from a target to be imaged in response to exposure to the pulses of light, and to convert the ultrasonic waves to a digital signal. The signal processor may be configured to perform image processing on the digital signal to determine image data for a 1D, 2D, or 3D image of the target. For example, the image processing may involve any one or any combination of: beamforming processing, backprojection processing, and/or various techniques for inversion processing. In some aspects, the signal processor may be configured to determine depth information from the ultrasonic waves by performing any one or any combination of: analog filtering, digital filtering, deramp processing, cross-correlation processing, pulse-compression processing, and heterodyne processing.

According to aspects of the present technology, a photoacoustic imaging method is provided. The method may comprise scanning a target using a photoacoustic imaging probe comprising: an ultrasound-on-a-chip device that includes a plurality of ultrasonic transducers supported by a housing, and an optical emitter supported by the housing, the optical emitter comprising an array of diodes arranged at a periphery of the plurality of ultrasonic transducers; and performing signal processing on photoacoustic ultrasonic waves generated by the target to convert the ultrasonic waves to a digital signal used to produce a photoacoustic image. The scanning may comprise: irradiating the target with light from the array of diodes, and detecting the photoacoustic ultrasonic waves at the plurality of ultrasonic transducers.

In a variation of this aspect, the signal processing may comprise image formation (e.g., using beamforming techniques) to create one or more spatial map(s) of the photoacoustic ultrasonic waves generated by the target, and may also comprise any one or any combination of: deramp processing, cross-correlation processing, pulse-compression processing, and heterodyne processing to determine depth information from the photoacoustic ultrasonic waves.

In another variation of this aspect, the irradiating of the target may comprise irradiating the target with chirped pulses of light from the array of diodes.

In another variation of this aspect, the method may further comprise: performing a background scan of the target by emitting ultrasonic waves from the plurality of ultrasonic transducers and receiving back-scattered ultrasonic waves from the target; processing the back-scattered ultrasonic waves from the target to produce an ultrasound image; and comparing the ultrasound image with the photoacoustic image to determine features of the target that appear in one but not both of the ultrasound image and the photoacoustic image, or in both of these images.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIGS. 1A, 1B, and 1C show different photoacoustic imaging procedures enabled by photoacoustic imaging apparatuses, according to some embodiments of the technology described herein;

FIG. 2A shows a block diagram of a photoacoustic imaging system, according to some embodiments of the technology described herein;

FIG. 2B shows a block diagram of an ultrasound system that may be incorporated in the photoacoustic imaging system of FIG. 2A, according to some embodiments of the technology described herein;

FIG. 3 is a block diagram of a photoacoustic imaging system, according to some embodiments of the technology described herein;

FIG. 4 is a block diagram of a photoacoustic device that may be employed in the photoacoustic imaging system of FIG. 3, according to some embodiments of the technology described herein;

FIG. 5 is a block diagram illustrating how components of the photoacoustic device of FIG. 4 may be used, according to some embodiments of the technology described herein;

FIGS. 6, 7, and 8 schematically show different configurations of a photoacoustic imaging probe, according to embodiments of the technology described herein; and

FIGS. 9, 10, and 11 show block diagrams for different signal processors that may be used to process signals output from ultrasonic transducers, according to some embodiments of the technology described herein.

### DETAILED DESCRIPTION

Photoacoustics involves the formation of sound waves by a material through absorption of light by the material. The absorbed light causes pressure changes in the material, which may be ultrasonic pressure changes that may be detectable by an ultrasonic transducer.

Conventional photoacoustic systems often use a high-power Q-switched laser as a source of light, with the light being delivered to an imaging target via a fiber optical output. The laser typically is a diode-pumped solid-state (DPSS) laser operated to emit energy in the form of light onto the target in one or more pulse(s) of a high peak power. For example, the emitted energy for one pulse may be of the order of several milliJoules (mJ) lasting for several nanoseconds (ns), resulting in a peak power in the megawatt (MW) range. The laser may be operated at a repetition rate that is power-limited to tens or hundreds of Hz. Because such lasers need not have a long cavity length, they can be as small as a large laser pointer. However, in applications suitable for photoacoustic imaging, the ability to tune or adjust the wavelength of the emitted energy is highly desirable, which tends to add bulk and cost to systems that use such lasers.

More specifically, in photoacoustic imaging applications, the ability to use a desired output wavelength of light to be directed at an imaging target can be highly beneficial for enabling different materials to be identified in a photoacoustic image. For example, one type of animal tissue (e.g., blood-vessel tissue) may exhibit a particular absorption characteristic for a first color or wavelength of light, while another type of animal tissue (e.g., muscle tissue) may exhibit a particular absorption characteristic for a second color or wavelength of light and may not efficiently absorb light of the first wavelength. Photoacoustic imaging may enable the existence and locations of different materials in a target (e.g., the presence and location of blood vessels, a tumor, bone, etc., in a leg muscle of the target) to be discerned. By sequentially irradiating the target with light of different wavelengths, to produce the photoelectric effect in the target under different light-absorption conditions, a detailed image may be produced from the ultrasound waves emitted by the target. As will be appreciated, a material may exhibit an absorption peak (or valley) for a range of wavelengths of light; therefore, reference herein to a wavelength of light at which absorption may occur (e.g., absorption at 650 nm) may comprise a range encompassing that wavelength (e.g., a range of 550 nm to 800 nm). Optionally, simultaneous irradiation with light of different wavelengths may be performed; however, more complex signal processing (e.g., using any one or any combination of: Hadamard codes, orthogonal waveforms, inverted chirps, reversed chirps, etc.) would be required if depth information is to be extracted from the ultrasonic waves produced by the target.

With conventional photoacoustic imaging systems, wavelength tuning may involve the use of an optical parametric oscillator (OPO), which may be a combination of a nonlinear crystal and a cavity to convert from one wavelength to other wavelengths. Use of a nonlinear crystal may be, in some respects, analogous to use of a doubling crystal to achieve frequency doubling in a DPSS laser, although other complexities may arise from the additional use of the cavity with the nonlinear crystal. As will be appreciated, conventional photoacoustic systems with wavelength tunability can be costly and bulky.

The inventors have appreciated the need for a photoacoustic imaging system that is compact enough to be hand held, that can be portable, that can be produced at a cost significantly lower than that of a conventional photoacoustic imaging system, and that can readily allow multiple wavelengths to be used sequentially without requiring retooling or reconfiguration of the system. To this end, in some embodiments of the technology disclosed herein, a portable, battery-powered photoacoustic imaging system is provided, in which the system may be a probe that is sized to be held in a user's hand. The probe may include diodes for illuminating a target with light to produce the photoacoustic effect, and ultrasonic transducers for detecting ultrasonic waves emitted from the target. As described in more detail below, the probe may be structured to include a housing that accommodates the diodes, the ultrasonic transducers, a power source (e.g., a battery), and other electronic circuitry, and may include a handle that can be grasped easily by the user's hand during imaging of the target.

In some embodiments of the present technology, the diodes may be an array of light-emitting diodes (LEDs) or laser diodes or a combination of LEDs and laser diodes. The diodes may be arranged on a head portion of the probe, such that when the probe is held in the user's hand the diodes may emit light toward the target. The transducers may be MEMS devices formed on a semiconductor chip that also may include signal-processing circuitry and other circuitry integrated onto the same chip (e.g., ultrasound-on-a-chip devices). Different types of MEMS transducers are discussed below. The transducers may be arranged on the head portion of the probe such that, when the probe is held in the user's hand to may emit light toward the target, the transducers may receive ultrasound waves photoacoustically generated by the target in response to the light. The transducers may be arranged in a detector region on the head portion of the probe, and the diodes may be distributed at a periphery of the detector region. For example, the diodes may be arranged at one or more section(s) at the periphery of the detector region, or may completely surround the periphery of the detector region. With such arrangements, the probe may emit light and receive ultrasonic waves while held in a single position relative to the target or while being moved or scanned relative to the target. Processing of the received waves may be performed in real time by the on-chip circuitry.

Unlike a Q-switched DPSS laser, which typically can emit light at a relatively high power (e.g., an optical peak power in the 10⁶ W or MW range), diodes typically can emit light at a relatively low power (e.g., an optical peak power in the W range).

In the case of Q-switched lasers, a single high-power pulse may be used effectively to illuminate a target, as done conventionally for photoacoustic imaging. More specifically, a Q-switched DPSS laser may be suitable for obtaining an image from a higher-power pulse of 200 kW/cm² having a duration of 100 ns or shorter. For example, a single pulse may be used to illuminate an entire volume of over ten cm³ simultaneously, to generate acoustic pressure in the 10⁶ Pa or MPa range.

Diodes may enable an image to be obtained from a relatively longer pulse of 1 millisecond (ms) at a power of 200 W/cm² or lower. With such a longer pulse, the possibility of detecting ultrasonic waves originating from deeper in the target is possible, therefore enabling imaging of a deeper volume within the target. For example, in contrast to Q-switched DPSS lasers, diodes can generate acoustic pressure in the target of only a few Pa, which results in an even lower pressure when the ultrasonic waves propagate and expand at the transducers. Sensing or detection of the lower-pressure ultrasonic waves by the transducers may require the diodes to emit a relatively long pulse train at a frequency of a few MHz. When probing deep tissue of the target or probing through the target's skull, or when lower acoustic pulse attenuation is desired, a lower repetition rate may be used, e.g., in the range of 100 kHz. For noise considerations, i.e., to obtain a lower noise, the pulse train may be averaged over a duration of tens of ms. In some embodiments of the technology described herein, the diodes may emit chirped optical pulses. Properties of the target (e.g., surface terrain, feature depth, tissue type, etc.) may be determined based on frequency analysis and/or phase analysis of the chirped optical/acoustic pulses or via acoustic frequency scans (e.g., at 1 MHz to 10 MHz). For example, optical pulsing may occur at a rate of 1 MHz to 10 MHz. An imaging response rate of tens of Hz is possible for photoacoustic imaging using diodes.

The array of diodes may include diodes having a distribution of power. The relatively lower power of the diodes, in comparison with a Q-switched DPSS laser, enables a noise reduction of approximately 40% to 50%. For example, when using resonant piezoelectric transducers for detection, the signal-to-noise ratio (SNR) may be 77 dB when a Q-switched DPSS laser is used for light emission, and the SNR may be 43 dB when diodes are used for light emission. Noise may not be a significant issue with certain transducers that can resolve signals on the order of 1 mPa/Hz^{1/2}.

The array of diodes may include diodes of different color, i.e., that emit light having different wavelengths. Using diodes of different color for photoacoustic imaging may facilitate color tuning, where the different wavelengths or colors may be selectively emitted by powering different diodes selectively. The variations that may be observed by comparing images obtained using different wavelengths of light may reveal details about the target that may not be readily observable in a photoacoustic image obtained at a single wavelength, due the absorption differences discussed above. As previously mentioned, the diodes of different color may be powered sequentially, one color or a few colors at a time (e.g., in order by wavelength magnitude(s)), or the diodes may be powered simultaneously.

The round-trip time from launching a light pulse to detection of the acoustic pressure generated by the target (i.e., detection of the ultrasonic waves by the transducers) may be used as an indication of the depth within the target from which the acoustic pressure was generated. The term “photoacoustic waves” may be used herein to refer to ultrasonic waves generated by the photoelectric effect. Three-dimensional (3D) images of the target may be obtained through appropriate processing of the ultrasonic waves received by the transducers in response to the emitted light. Ultrasonic waves may be generated from a volume within the target, which may extend from the surface of the target to a depth below the surface of the target. The relative depths from which the ultrasonic waves are generated may be determined from round-trip time measurements starting from time of the emission of the light waves to the time of detection of the photoacoustically generated ultrasonic waves by the transducers, and by signal processing techniques that use information about the emitted signals and information about the received signals.

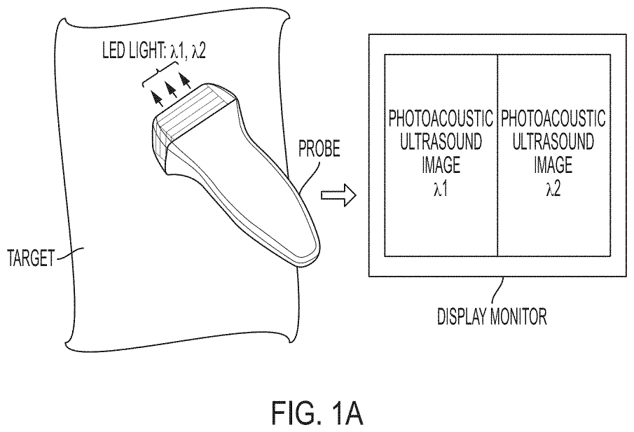

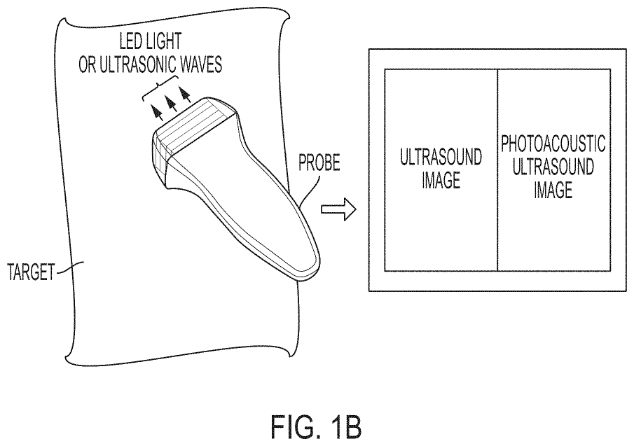

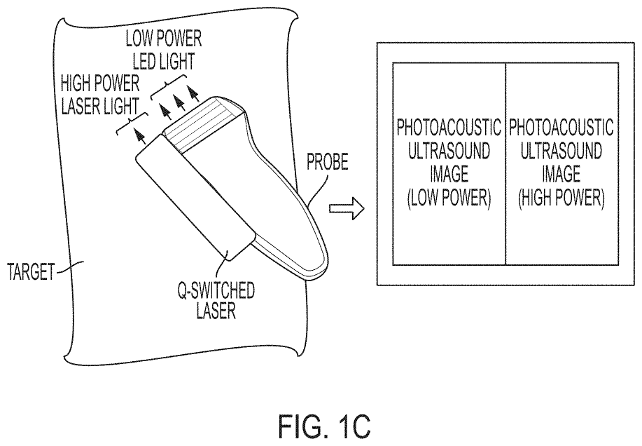

FIGS. 1A, 1B, and 1C show different photoacoustic imaging implementations enabled by photoacoustic imaging apparatuses discussed below, according to some embodiments of the technology described herein.

In the implementation shown in FIG. 1A, a photoacoustic probe may be used to irradiate a target with LED light of two different wavelengths λ1, λ2 to obtain a photoacoustic ultrasound image corresponding to λ1 and a photoacoustic ultrasound image corresponding to λ2. These images may be 1D, 2D, or 3D images, and may be displayed on a display monitor during real-time (or nearly real time) imaging. Because light absorption by different target materials may be different, the two images may yield different information, which may provide medically useful information regarding the target.

In the implementation shown in FIG. 1B, a photoacoustic probe may be used to irradiate a target with LED light to obtain a photoacoustic ultrasound image of the target, and to irradiate the target with ultrasonic waves (e.g., ultrasonic waves generated by transducers of the probe) to obtain an ultrasound image. As will be appreciated, the ultrasound image results from back-scattered ultrasonic waves, which reflect from surfaces and interfaces of the target, and typically does not yield information about a volume of the target beyond the reflecting surface.

The photoacoustic ultrasound image, on the other hand, may be a 3D image of the volume of the target. These images may be displayed on a display monitor to enable a comparison of surface features and below surface features of, e.g., an organ of the target.

In the implementation shown in FIG. 1C, a photoacoustic probe may be used to irradiate a target with LED light to obtain a 1D, 2D, or 3D photoacoustic ultrasound image of the target, and to irradiate the target with high-power light from a Q-switched laser to obtain a 1D or 2D photoacoustic ultrasound image of the target. For example, the Q-switched laser may be attached to the probe such that an illumination region of the Q-switched laser is aligned with an illumination region of the LED light. These images may be displayed on a display monitor to enable a comparison of, e.g., 2D image features, which result from relatively high-power light from the Q-switched laser, with 3D features, which result from relatively low-power LED light.

### Noise Considerations and Optical Considerations

As will be appreciated, useful images, especially images that may be used for medical diagnoses, typically require not only the ability to discern a particular feature from surrounding features but also the ability to discern a particular feature for imaging artifacts that may obscure the particular feature. A detected photoacoustic signal may include a component generated by an imaging target (“signal”) as well as a component resulting from imaging artifacts (“noise”). The ratio of this components, or SNR, may be used to assess image quality.

For optical signals, the SNR may be limited by the pulse width (duration) of the optical pulse(s) used for excitation of the target (i.e., to cause the photoacoustic effect to occur). Government standards may limit the amount of energy to which a human target may be exposed. (See, e.g., Laser Institute of America, American National Standard for Safe Use of Lasers, ANSI Z136.1-2000, American National Standards Institute Inc., New York, 2000) For example, a government standard may limit the permissible exposure to an optical pulse in the near-infrared (near-IR) region of the optical spectrum according to an expression given by Es=Es₀*T^{1/4}, where Es₀ is 1100 mJ/cm² and T is exposure time (in seconds). Thus, a typical pulse from a Q-switched DPSS laser, having a width or duration of 10 ns, would have a maximum safe limit for optical power of approximately 11 mJ/cm², which converts to 1.1 MW/cm² input to the target. In comparison, a longer pulse width of 10 ms from an LED or a laser diode would have a maximum safe limit for optical power of approximately 350 mJ/cm², which converts to 35 W/cm² for a 10 ms pulse input to the target. By irradiating the target using an optical pulse having a longer pulse width at a much lower optical power (e.g., by using diodes instead of a Q-switched DPSS laser), while remaining within the limit(s) set by government standards, it is possible to cause the target to generate a relatively larger total acoustic signal. The longer pulses permitted at the lower power of a diode may be used to probe deeper into the target.

More specifically, in photoacoustics, the pressure induced or generated at the target is directly proportional to the power absorbed by the molecules of the target. As such, the SNR in a photoacoustic imaging system may be proportional to P²/N, where P is the optical power density and roughly is Es/T, and where N is the noise limit of the acoustic detector (e.g., the ultrasound transducers). This noise limit may vary as a function of the square root of an averaging time (e.g., an average pulse width).

As discussed above, it is feasible to irradiate the target with light at or near the safety limit by using a short pulse (˜100 ns) from a Q-switched DPSS laser switched laser or by using a longer pulse (˜1 ms) from diodes. Therefore, before accounting for attenuation in media, the SNR may be determined as the square of the optical power density P relative to the noise limit of acoustic detector N. If the square of the noise limit is defined by (or estimated to be) N²=No/T, and if the optical power density is defined by or estimated to be P=E/T, then the SNR may be determined according to the following expression:

SNR=(P/N)²=((E/T)/N)²=(Es₀*T^{1/4})/T)²/(N₀/T)=Es₀²/(N₀T^{1/2}),

where No is a nominal noise limit.

As will be appreciated from the expression above, the SNR drops as the square root of the inverse of the pulse width. Therefore, for light from a Q-switched laser with a pulse width of ˜100 ns, a good match to a 5 MHz acoustic detector will have a SNR that is 100 times better than light from diodes with a pulse width of 1 ms (based on the square root of the 10,000-fold ratio of the pulse widths). With diodes, 1 ms pulses of light at 10 Hz may have a lower SNR by about 40 dB. However, it is possible to recover 20 dB through use of a more resonant acoustic detector having a 20 dB improved response. Accordingly, with diodes, 1 ms pulses of light at 10 Hz is expected to have about a 20 dB lower SNR compared with a Q-switched laser.

For continuous irradiation with diodes at a modulation of 10 Hz, the peak power for safe operation is limited to 0.2 W/cm² and lower. Under this limitation, and at a relatively lower 10 Hz bandwidth for reduced noise, a diode-based photoacoustic imaging system may have a 60 dB lower SNR than a system based on a Q-switched laser and a 100 ns pulse width. However, the SNR can be improved to 40 dB lower than for the Q-switched system operated at 100 ns by using a resonant acoustic detector having an improved sensitivity.

When the target is irradiated with longer pulse widths, uniquely identifying a signal's depth of origin within the target (i.e., the depth from which the detected signal originated) may not be a simple task. For instance, a 5 MHz oscillation or frequency at a sound speed of 1.5 km/s will have a wavelength of 0.3 mm. As such, a continuous wave cannot be used to resolve the position of a signal within this periodicity. However, the position can be resolved by using a chirped or frequency-scanned pulse train.

With a chirped or frequency-scanned pulse train, different depths of origin will have a different phase of the chirped or frequency-scanned signal. An axial resolution of such a pulse train can be scanned over a frequency bandwidth Δv during emission of the pulse train. Signal processing of ultrasound signals generated by such pulse trains is described below and may be performed using one or more array(s) of MEMS transducers (e.g., capacitive micromachined ultrasonic transducers (CMUTs)) configured to allow simultaneous imaging of a volume of the target in its entirety using parallel software channels.

SNR may be increased for a diode-based photoacoustic imaging system by using cross-correlation signal-processing techniques via a product of time and bandwidth. For example, a 1 ms chirped pulse train swept from 500 kHz to 15 MHz or with a bandwidth centered at 1 MHz may have an increase in signal power by nearly three orders of magnitude (taking into consideration the travel time has an optical contribution and an acoustic contribution). Through chirping, all the light energy is output into a smaller time resolution, and in some cases the cross correlation of the signals may increase the SNR nearly proportionally. Moreover, as mentioned above, the safety limit for a 1 ms pulse width outputted by diodes (e.g., 200 mJ/cm²) has an energy value that is ten times higher than the safe limit for a 100 ns pulse width outputted by a Q-switched laser (e.g., 20 mJ/cm²), so it is possible to have a better SNR for a diode-based system. In practice, however, real-time imaging at ˜10 Hz is desirable and therefore an average power of 0.2 W/cm² at 10 Hz limits the energy to about 20 mJ/cm², regardless of the duration of the pulse width, i.e., continuous operation is possible at this level of power.

### System Architectures

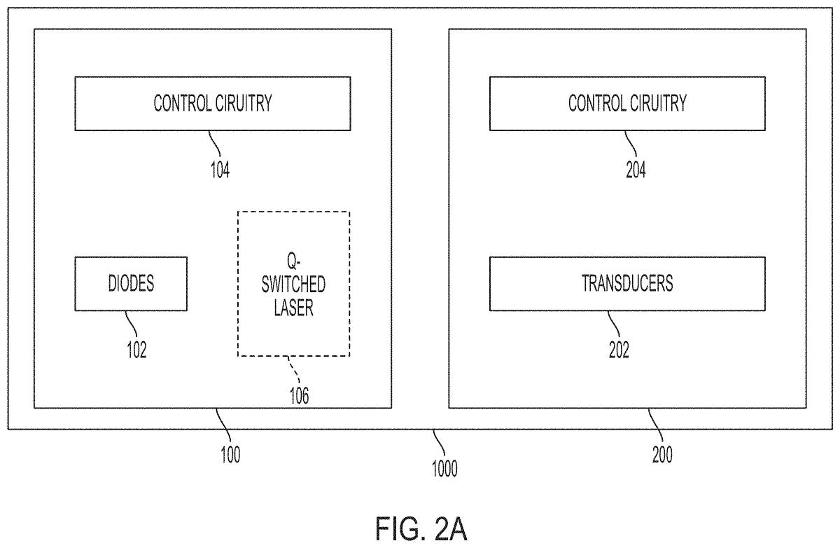

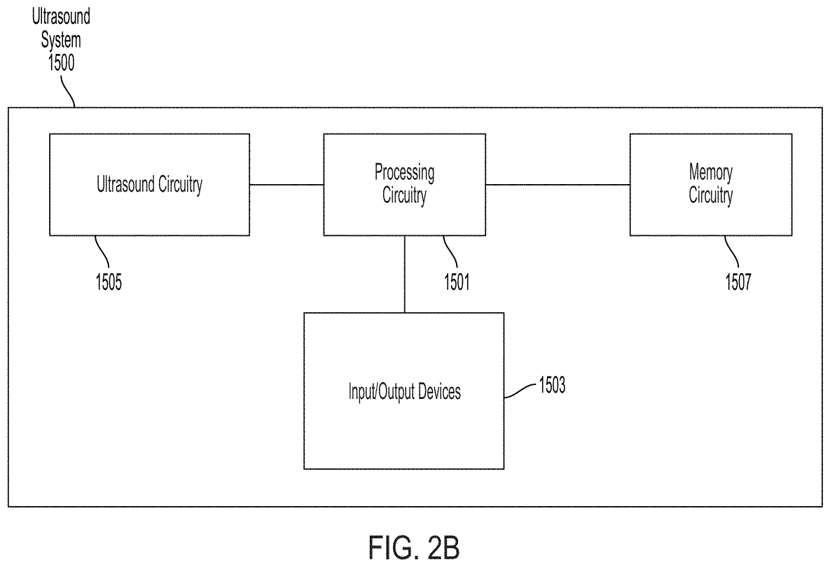

FIG. 2A is a block diagram of a photoacoustic imaging system 1000, according to some embodiments of the technology described herein. The system 1000 may include an optical device 100 and an acoustic device 200.

The optical device 100 may include diodes 102, which may include light-emitting diodes (LEDs), or laser diodes, or a combination of LEDs and laser diodes. The diodes 102 may have wavelengths in the visible range (roughly 400 nm to 690 nm) and/or wavelengths in the near infrared (NIR) range (roughly 700 nm to 2000 nm). In some embodiments of the present technology, the diodes 102 may include one or more array(s) of LEDs having a single wavelength (e.g., only red LEDs having a peak wavelength of 645 nm, or only amber LEDs having a peak wavelength of 610 nm, or only green LEDs having a peak wavelength of 523 nm, etc.). In some embodiments, the diodes 102 may include one or more array(s) of LEDs having two or more different wavelengths (e.g., any combination of red LEDs, amber LEDs, green LEDs, NIR LEDs, etc.). In some embodiments, the diodes 102 may include one or more array(s) of laser diodes having a single wavelength or having two or more different wavelengths. In some embodiments, the diodes 102 may include a combination of LEDs and laser diodes, and the diodes may have a single wavelength or may have two or more different wavelengths.

As will be appreciated, a “wavelength” of a diode refers to a peak wavelength of the diode. Also as will be appreciated, an LED having a particular wavelength will have a relatively wider spectral width or spread on both sides of the peak, and a laser diode will have a relatively narrower spread on both sides of the peak. For example, an LED can have a spread on the order of tens of nm, and a laser diode can have a spread on the order of a few nm.

In some embodiments of the present technology, diodes of one wavelength may be segregated relative to diodes of another wavelength. In some other embodiments, diodes of different wavelengths are interspersed with each other.

The optical device 100 may include control circuitry 104 that controls the emission of light from the photoacoustic imaging system 1000. The control circuitry 104 may include circuitry for generating an optical pulse and for controlling a pulseform of the optical pulse.

Optionally, in addition to the diodes 102, the optical device 100 may include one or more Q-switched laser(s) 106, which may provide the photoacoustic imaging system 1000 the ability to compare high-optical-power images (obtained with the laser(s) 106) with low-optical-power images (obtained with the diodes 102). For example, the laser(s) 106 may include a 660 nm Nd:YAG laser and/or a 532 nm Nd:YAG laser. Operation of the laser(s) 106 may be controlled by the control circuitry 104.

The acoustic device 200 may comprise transducers 202 configured to detect ultrasonic waves generated by a target in response to irradiation of light from the optical device 100. The transducers 202 also may be configured to emit ultrasonic waves. For example, it may be desirable to obtain an ultrasound image of the target as an initial or baseline image. The transducers 202 may be used to perform an ultrasound scan by transmitting and detecting ultrasonic waves. The acoustic device 200 may include control circuitry 204 that controls the emission of ultrasonic waves generated by the transducers 202 as well as the detection and processing of ultrasonic waves by the transducers 202.

FIG. 2B is a block diagram illustrating aspects of an example ultrasound system 1500 that may form at least a part of the acoustic device 200. As shown, the ultrasound system 1500 may comprise processing circuitry 1501, input/output devices 1503, ultrasound circuitry 1505, and memory circuitry 1507.

The ultrasound circuitry 1505 may be configured to generate ultrasound data that may be employed to generate a photoacoustic image. The ultrasound circuitry 1505 may comprise ultrasonic transducers monolithically integrated onto a single semiconductor die. The ultrasound circuitry 1505 may, for instance, be micromachined devices fabricated using semiconductor processing technology. The transducers may include any combination of one or more CMUT(s), one or more CUT(s), one or more PMUT(s), and/or one or more other suitable ultrasonic transducer cells. In some embodiments of the technology described herein, the ultrasonic transducers may be formed on the same chip as other electronic components in the ultrasound circuitry 1505 (e.g., transmit circuitry, receive circuitry, control circuitry, power management circuitry, and processing circuitry) to form a monolithic ultrasound device. For example, on-chip signal processing may be performed in the signal path of received ultrasonic signals (e.g., to reduce data bandwidth) and/or a high-speed serial data module may be used to move digitized data off-chip (e.g., as a digital data stream). The digitization of received ultrasonic signals on-chip may allow other on-chip processing, such as advanced digital signal processing (DSP), to be performed on-chip. In some implementations, such on-chip DSP may permit complete or substantially complete integration of an entire ultrasonic imaging system on a single semiconductor substrate. In some other implementations, a high-speed stream of digital data may be output from the chip to an external computer (not shown) for further processing and/or display on a screen of the external computer; for instance, some image-formation processing may be performed on-chip and other image-formation processing may be performed on the external computer. As will be appreciated, image-processing functionality provided by the external computer may complement image-processing functionality provided on-chip. Ultrasound-on-a-chip technology is described in U.S. Pat. No. 9,521,991 issued Dec. 20, 2016, entitled “MONOLITHIC ULTRASONIC IMAGING DEVICES, SYSTEMS AND METHODS,” the entire contents of which is incorporated by reference.

The processing circuitry 1501 may be configured to perform any of the functionality described herein. The processing circuitry 1501 may comprise one or more processors (e.g., computer hardware processors). To perform one or more functions, the processing circuitry 1501 may execute one or more processor-executable instructions stored in the memory circuitry 1507. The memory circuitry 1507 may be used for storing programs and data for operation of the ultrasound system 1500 and for operation of the photoacoustic imaging system 1000. The memory circuitry 1507 also may store data obtained during operation of the ultrasound system 1500 and the photoacoustic imaging system 1000. The memory circuitry 1507 may comprise one or more storage devices, such as non-transitory computer-readable storage media. The processing circuitry 1501 may control writing data to and reading data from the memory circuity 1507 in any suitable manner.

In some embodiments of the present technology, the processing circuitry 1501 may comprise specially-programmed and/or special-purpose hardware such as an application-specific integrated circuit (ASIC). For example, the processing circuitry 1501 may comprise one or more signal processing units for routines for performing cross-correlation processing, chirp processing, Fourier-transformation processing (e.g., FFT), heterodyne processing, and other techniques for processing signals.

The input/output (I/O) devices 1503 may be configured to facilitate communication with other systems and/or an operator. Example I/O devices that may facilitate communication with an operator may include: a keyboard, a mouse, a trackball, a microphone, a touch screen, a printing device, a display screen, a speaker, and the like. Example I/O devices that may facilitate communication with other systems may include wired and/or wireless communication circuitry, such as BLUETOOTH, ZIGBEE, WiFi, USB communication circuitry, and the like.

It should be appreciated that the ultrasound system 1500 may be implemented using any number of devices. For example, the components of the ultrasound system 1500 may be integrated into a single device. In another example, the ultrasound circuitry 1505 may be integrated into an ultrasound device that is communicatively coupled with a computing device that comprises the processing circuitry 1501, the input/output devices 1503, and the memory circuitry 1507.

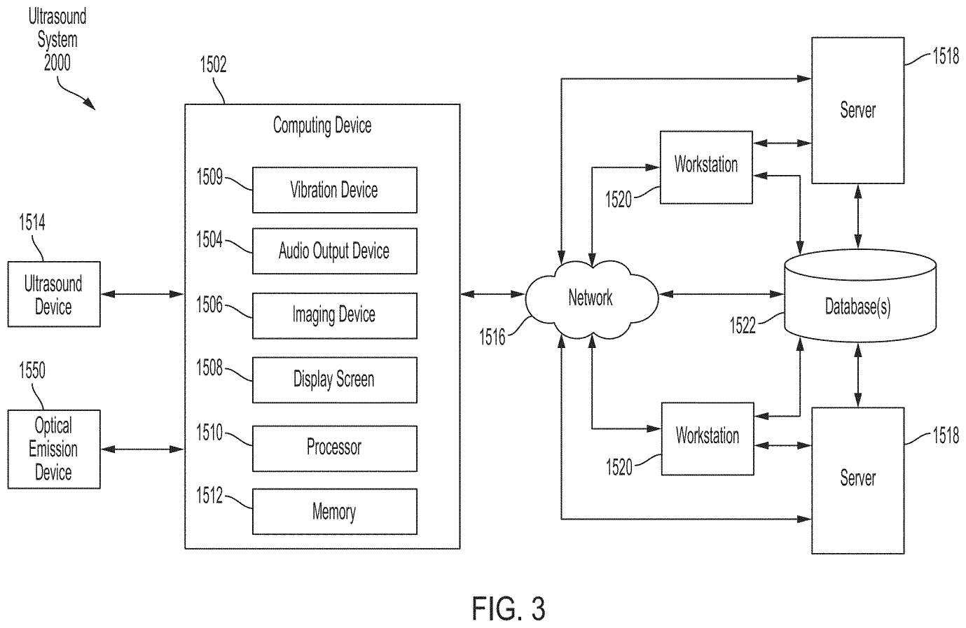

FIG. 3 is a block diagram of a photoacoustic imaging system 2000 according to some embodiments of the technology described herein. For example, one or more components of the photoacoustic imaging system 2000 may perform any of the processes described herein. As shown, the photoacoustic imaging system 2000 may comprise an ultrasound device 1514 and an optical emission device 1550 in wired and/or wireless communication with a computing device 1502. The computing device 1502 may comprise an audio output device 1504, an imaging device 1506, a display screen 1508, a processor 1510, a memory 1512, and a vibration device 1509. The computing device 1502 may communicate with one or more external devices over a network 1516. For example, the computing device 1502 may communicate with one or more workstations 1520, servers 1518, and/or databases 1522.

The optical emission device 1550 may include a light source and transmit circuitry that controls the light source to emit light having a desired form and/or a desired one or more wavelength(s). The light source may comprise diodes (e.g., LEDs and/or laser diodes, such as those discussed above), and the diodes may emit light of different colors corresponding to different wavelengths. Optionally, in addition to the diodes, the light source may include one or more Q-switched laser(s) (e.g., Nd:YAG laser(s), such as those discussed above). For example, the transmit circuitry may control the light source to emit light pulses of light sequentially according to wavelength (e.g., shortest to longest, or longest to shorted), and may control a pulse width of the emitted light. The transmit circuitry also may control a chirping process, such that the pulses of emitted light are chirped pulses.

The ultrasound device 1514 may be configured to receive ultrasonic waves photoacoustically generated by the target, and to generate ultrasound data that may be employed to generate a photoacoustic image of the target. The ultrasound device 1514 also may be configured to transmit ultrasonic waves to the target and receive back-scattered or reflected ultrasonic waves in return, and to generate ultrasound data that may be employed to generate an ultrasound image of the target. The ultrasound device 1514 may be constructed in any of a variety of ways. In some embodiments of the present technology, the ultrasound device 1514 may include transmit circuitry that controls a transmit beamformer to drive transducer elements within a transducer array to emit pulsed ultrasonic signals to the target, and may include receive circuitry that controls the transducer elements of the array to receive ultrasonic waves. For example, in cases where pulsed ultrasonic signals are emitted to the target, the received ultrasonic waves may result from the pulsed ultrasonic signals being back-scattered from structures in the target (e.g., blood vessels, muscular tissue, bone, etc.) to produce echoes that return to the transducer elements; alternatively, in cases where the target is irradiated with light (e.g., chirped pulses of light), the received ultrasonic waves may be photoacoustically generated by the structures in the target. The received ultrasonic waves may then be converted into electrical signals (photoacoustic signals) by the transducer elements. Subsequently, these electrical signals may be sent to a receive beamformer that outputs ultrasound data used to produce an image of the target.

The computing device 1502 may be configured to process the ultrasound data from the ultrasound device 1514 to generate ultrasound images for display on the display screen 1508. The processing may be performed by, for example, the processor 1510. The processor 1510 may also be adapted to control the acquisition of ultrasound data with the ultrasound device 1514. The ultrasound data may be processed in real-time during a scanning session as the ultrasonic signals are received. In some embodiments of the present technology, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data may be sequentially displayed. Additionally, or alternatively, the ultrasound data may be stored temporarily in a buffer during a scanning session and processed in less than real-time.

Additionally (or alternatively), the computing device 1502 may be configured to perform any of the processes described herein (e.g., using the processor 1510) and/or to display a user interface (e.g., using the display screen 1508). For example, the computing device 1502 may be configured to provide instructions to an operator of the ultrasound device 1514 to assist the operator select an anatomical view of the target to image, and to guide the operator to capture an ultrasound image containing the anatomical view. As shown, the computing device 1502 may comprise one or more elements that may be used during the performance of such processes. For example, the computing device 1502 may comprise one or more processors 1510 (e.g., computer hardware processors) and one or more articles of manufacture that comprise non-transitory computer-readable storage media, such as the memory 1512. The processor 1510 may control writing data to and reading data from the memory 1512 in any suitable manner. To perform any of the functionality described herein, the processor 1510 may execute one or more processor-executable instructions stored in one or more non-transitory computer-readable storage media (e.g., the memory 1512), which may serve as non-transitory computer-readable storage media storing processor-executable instructions for execution by the processor 1510.

Portions of the photoacoustic imaging system 2000 may be sized to be held in an operator's hand, such that that the operator may be the target and may use the photoacoustic imaging system 2000 to obtain photoacoustic images of the operator's own body.

In some embodiments of the present technology, the computing device 1502 may comprise one or more input and/or output devices, such as the audio output device 1504, the imaging device 1506, the display screen 1508, and the vibration device 1509. The audio output device 1504 may be a device that is configured to emit audible sound such as a speaker. The imaging device 1506 may be a camera. The display screen 1508 may be configured to display images and/or videos such as a liquid crystal display (LCD), a plasma display, and/or an organic light emitting diode (OLED) display. The vibration device 1509 may be configured to vibrate one or more components of the computing device 1502 to provide tactile feedback. These input and/or output devices may be communicatively coupled to the processor 1510 and/or under the control of the processor 1510. The processor 1510 may control these devices in accordance with a process being executed by the processor 1510. For example, the processor 1510 may control the display screen 1508 to display a user interface, instructions, and/or ultrasound images. Similarly, the processor 1510 may control the audio output device 1504 to issue audible instructions and/or control the vibration device 1509 to change an intensity of tactile feedback (e.g., vibration) to issue tactile instructions. Additionally (or alternatively), the processor 1510 may control the imaging device 1506 to capture photographs of the target prior to or during a photoacoustic imaging session.

It should be appreciated that the computing device 1502 may be implemented in any of a variety of ways. For example, the computing device 1502 may be implemented as a handheld device (e.g., a mobile smartphone, a tablet, and the like). Thereby, the operator of the ultrasound device 1514 may be able to operate the ultrasound device 1514 with one hand and hold the computing device 1502 with another hand. In other examples, the computing device 1502 may be implemented as a portable device that is not a handheld device such as a laptop. In yet other examples, the computing device 1502 may be implemented as a stationary device, such as a desktop computer.

In some embodiments, the computing device 1502 may communicate with one or more external devices via the network 1516. The computing device 1502 may be connected to the network 1516 over a wired connection (e.g., via an Ethernet cable) and/or a wireless connection (e.g., over a WiFi network). As shown in FIG. 3, these external devices may include servers 1518, workstations 1520, and/or databases 1522. The computing device 1502 may communicate with these devices to, for example, off-load computationally intensive tasks. For example, the computing device 1502 may send an ultrasound image over the network 1516 to the server 1518 for analysis (e.g., to identify an anatomical feature in the ultrasound image and/or identify an instruction to provide the operator) and receive the results of the analysis from the server 1518. Additionally (or alternatively), the computing device 1502 may communicate with these devices to access information that is not available locally and/or update a central information repository. For example, the computing device 1502 may access the medical records of a subject being imaged with the ultrasound device 1514 from a file stored in the database 1522. In this example, the computing device 1502 may also provide one or more captured ultrasound images of the subject to the database 1522 to add to the medical record of the subject.

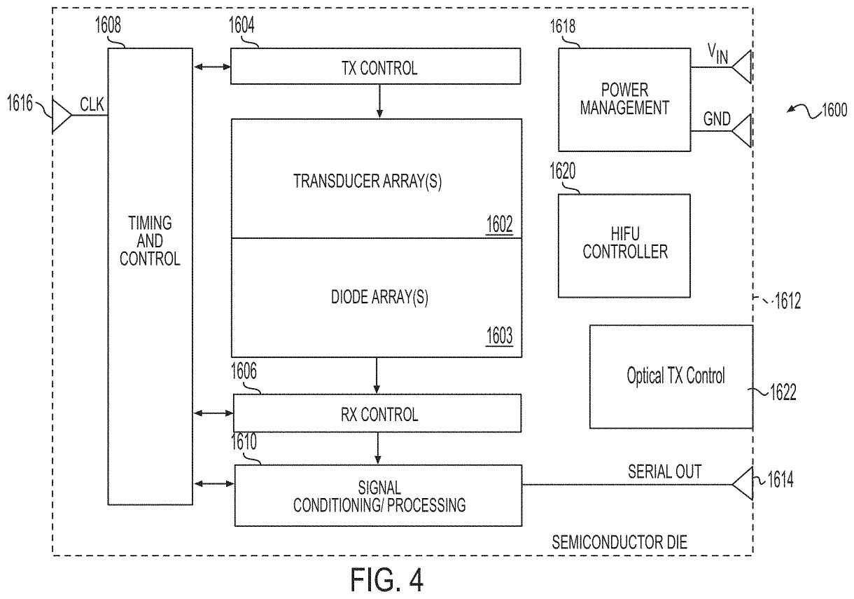

FIG. 4 is a block diagram of a photoacoustic device 1600 that may be employed as the ultrasound device 1514 and the optical emission device 1550 discussed above. As shown, the photoacoustic device 1600 may include one or more transducer array(s) 1602, one or more diode array(s) 1603, transmit (TX) control circuitry 1604, receive (RX) control circuitry 1606, a timing and control circuit 1608, a signal conditioning/processing circuit 1610, a power management circuit 1618, and/or a high-intensity focused ultrasound (HIFU) controller 1620. In the embodiment shown in FIG. 4, all of the illustrated components are formed on a single semiconductor die 1612. It should be appreciated, however, that in alternative embodiments, one or more of the illustrated components may instead be located off-chip. In addition, although the illustrated example shows both TX control circuitry 1604 and RX control circuitry 1606, in alternative embodiments only TX circuitry or only RX control circuitry may be employed. For example, such alternative embodiments may be employed in circumstances where a separate transmission-only device is used to transmit optical and/or acoustic signals to the target, or a separate reception-only device is used to receive acoustic signals from the target.

It should be appreciated that communication between one or more of the illustrated components may be performed in any of numerous ways. In some embodiments of the present technology, for example, one or more high-speed busses (not shown), such as that employed by a unified Northbridge, may be used to allow high-speed intra-chip communication or communication with one or more off-chip components.

The one or more transducer array(s) 1602 may take on any of numerous forms, and aspects of the present technology do not necessarily require the use of any particular type or arrangement of transducer elements. The one or more diode array(s) 1603 may take on any of numerous forms, and aspects of the present technology do not necessarily require the use of any particular type or arrangement of diodes. It should be appreciated that the term “array” may refer to an organized pattern of elements or may refer to a random distribution of elements. In various embodiments of the present technology, the transducer elements of the transducer array(s) 1602 may be formed on the same chip as electronic components of the TX control circuitry 1604 and/or the RX control circuitry 1606, and may be integrated in a single so-called ultrasound-on-a-chip device. In some embodiments, the single device may be a handheld device. In various embodiments of the present technology, the diodes of the diode array(s) 1603 also may be formed on the same chip as the electronic components of the TX control circuitry 1604 and/or the RX circuitry control 1606, and may be integrated in the single ultrasound-on-a-chip device. The TX circuitry 1604 may, for example, generate pulses that drive the diodes of the diode array(s) 1603 (as a group or as subgroups sorted according to wavelength) so as to generate light pulses for causing generation of photoacoustic waves in the target, to be used for imaging the target. The TX control circuitry 1604 optionally may be used to generate pulses that drive the transducers of the transducer array(s) 1602 so as to acoustic signals to be used for imaging the target. Optionally, the diode array(s) 1603 may be controlled by optical TX control circuitry 1622, which may only control the diodes of the diode array(s) 1603 and may not function to control the transducers of the transducer array(s) 1602. The RX control circuitry 1606, on the other hand, may receive and process electronic signals generated by the transducers of the transducer array(s) 1602 when ultrasonic waves (photoacoustically generated acoustic signals or back-scattered acoustic signals) impinge upon the transducers.

In some embodiments of the present technology, the timing and control circuit 1608 may, for example, be responsible for generating all timing and control signals that are used to synchronize and coordinate the operation of the other elements in the photoacoustic device 1600.

The timing and control circuit 1608 may be driven by a single clock signal CLK supplied to an input port 1616. The clock signal CLK may, for example, be a high-frequency clock used to drive one or more of the on-chip circuit components. In some embodiments, the clock signal CLK may, for example, be a 1.5625 GHz clock or a 2.5 GHz clock used to drive a high-speed serial output device (not shown) in the signal conditioning/processing circuit 1610, or a 20 MHz clock or a 40 MHz clock used to drive other digital components on the semiconductor die 1612. The timing and control circuit 1608 may divide or multiply the clock signal CLK, as necessary, to drive other components on the die 1612. In other embodiments, two or more clocks of different frequencies (such as those referenced above) may be separately supplied to the timing and control circuit 1608 from an off-chip source.

The power management circuit 1618 may, for example, be responsible for converting one or more input voltages V_{IN} from an off-chip source into voltages needed to carry out operation of components of the photoacoustic device 1600, and for otherwise managing power consumption within the photoacoustic device 1600. In some embodiments of the present technology, a single voltage (e.g., 12V, 80V, 100V, 120V, etc.) may be supplied to the photoacoustic device 1600 and the power management circuit 1618 may step that voltage up or down, as necessary, using a charge pump circuit or via some other DC-to-DC voltage-conversion mechanism. In other embodiments, multiple different voltages may be supplied separately to the power management circuit 1618 for processing and/or distribution to the other components of the photoacoustic device 1600.

As shown in FIG. 4, in some embodiments of the present technology, a HIFU controller 1620 may be integrated on the semiconductor die 1612 to enable generation of HIFU signals via one or more elements of the transducer array(s) 1602. In other embodiments, a HIFU controller for driving the transducer array(s) 1602 may be located off-chip, or even within a device separate from the photoacoustic device 1600. That is, the photoacoustic device may be part of an of ultrasound-on-a-chip HIFU system, and may utilize photoacoustically generated images to show a region of the target before and/or after a HIFU procedure. It should be appreciated, however, that some embodiments may not have any HIFU capabilities; in such cases the HIFU controller 1620 may be omitted.

One or more output port(s) 1614 may output a high-speed serial data stream generated by one or more components of the signal conditioning/processing circuit 1610. Such data streams may, for example, be generated by one or more USB 3.0 modules, and/or one or more 10 GB, 40 GB, or 100 GB Ethernet modules, integrated on the semiconductor die 1612. In some embodiments of the present technology, the signal stream produced on the output port(s) 1614 can be fed to a computer, a tablet, a smartphone, and the like, to generate and/or display two-dimensional, three-dimensional, and/or tomographic images. In some embodiments, image formation capabilities may be incorporated in the signal conditioning/processing circuit 1610, such that image data may be output at the output port 1614 as a stream of serial data.

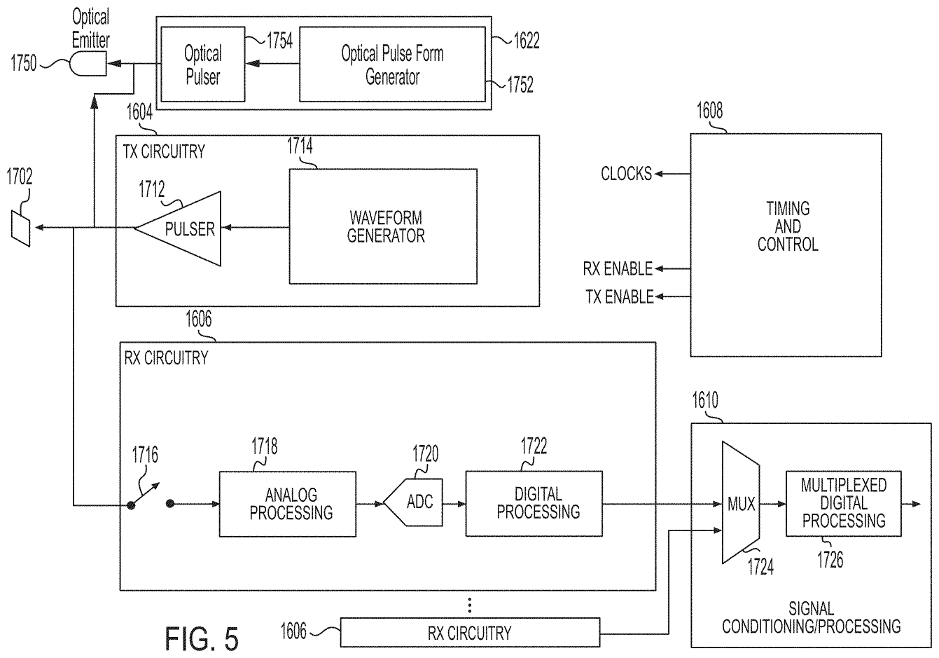

FIG. 5 is a block diagram illustrating how, in some embodiments of the technology described herein, the TX control circuitry 1604 and the RX control circuitry 1606 for a given one or more transducer element(s) 1702 may be used either to energize the transducer element(s) 1702 to emit an ultrasonic pulse, or to receive and process a signal from the transducer element(s) 1702 representing an ultrasonic pulse sensed by it, and illustrating how, in some embodiments, the optical TX control circuitry 1622 and/or the TX control circuitry 1604 may be used to energize a given one or more optical emitter(s) 1750 (e.g., LED(s), laser diode(s), etc.) to emit a light pulse. In some implementations, the TX control circuitry 1604 and/or the optical TX control circuitry 1622 may be used during a “transmission” (TX) phase, and the RX circuitry may be used during a “reception” (RX) phase, which may overlap or may be non-overlapping with the TX phase. For instance, the TX and RX phases may overlap when the optical TX control circuitry controls the emission of light by the optical emitter(s) 1750 and the RX circuitry controls the reception of ultrasonic signals. The TX control circuitry 1604 and/or the RX control circuitry 1606 may include a TX circuit and/or an RX circuit associated with a single transducer (e.g., a CUT or CMUT), a group of two or more transducers within a single transducer element 1702, a single transducer element 1702 comprising a group of transducers, a group of two or more transducer elements 1702 within an array 1602, or an entire array 1602 of one or more transducer element(s) 1702.

In the example shown in FIG. 5, the TX control circuitry 1604/RX control circuitry 1606 combination may include a separate TX circuit and a separate RX circuit for each transducer element(s) 1702 in the array(s) 1602, but there is only one instance of each of the timing and control circuit 1608 and the signal conditioning/processing circuit 1610. Accordingly, in some embodiments of the present technology, the timing and control circuit 1608 may be responsible for synchronizing and coordinating the operation of all of the TX control circuitry 1604/RX control circuitry 1606 combinations on the die 1612, and the signal conditioning/processing circuit 1610 may be responsible for handling inputs from all of the RX control circuitry 1606 on the die 1612. In other embodiments, the timing and control circuit 1608 may be replicated for each transducer element 1702 or for a group of transducer elements 1702.

As shown in FIG. 5, in addition to generating and/or distributing clock signals to drive the various digital components in the photoacoustic device 1600, the timing and control circuit 1608 may output either an “TX enable” signal to enable the operation of each TX circuit of the TX control circuitry 1604, or an “RX enable” signal to enable operation of each RX circuit of the RX control circuitry 1606. In the example shown, a switch 1716 in the RX circuitry 1606 may always be opened before the TX circuitry 1604 is enabled, so as to prevent an output of the TX circuitry 1604 from driving the RX circuitry 1606, when the TX circuitry 1604 is used to drive the transducer element(s) 1702 to emit ultrasonic pulses. The switch 1716 may be closed when operation of the RX circuitry 1606 is enabled, so as to allow the RX circuitry 1606 to receive and process a signal generated by the transducer element(s) 1702. The switch 1716 may remain indefinitely closed when the transducer element(s) 1702 are not used to emit ultrasonic pulses and the TX circuitry 1604 or the optical TX control circuitry 1622 control the optical emitter(s) 1750 to emit light pulses.

The TX control circuitry 1604 for a given one or more optical emitter(s) 1750 (or a given one or more transducer element(s) 1702) may include both a waveform generator 1714 and a pulser 1712. The waveform generator 1714 may, for example, be responsible for generating a waveform that is to be applied to the pulser 1712, so as to cause the pulser 1712 to output a driving signal to the optical emitter(s) 1750 (or to the transducer element(s) 1702) corresponding to the generated waveform. Similarly, the optical TX control circuitry 1622 for a given one or more optical emitter(s) 1750 may include both an optical pulse form generator 1752 and an optical pulser 1754. The form generator 1752 may, for example, be responsible for generating a pulse form that is to be applied to the optical pulser 1754, so as to cause the optical pulser 1754 to output a driving signal to the optical emitter(s) 1750 corresponding to the generated pulse form.

In the example shown in FIG. 5, the RX circuitry 1606 for a respective one or more transducer element(s) 1702 includes an analog processing block 1718, an analog-to-digital converter (ADC) 1720, and a digital processing block 1722. The ADC 1720 may, for example, comprise a 10-bit or 12-bit, 20 Msps, 25 Msps, 40 Msps, 50 Msps, or 80 Msps ADC.

After undergoing processing in the digital processing block 1722, the outputs of all of the RX circuits on the semiconductor die 1612 may be fed to the signal conditioning/processing circuit 1610. In embodiments where the number of RX circuits on the semiconductor die are equal a total number of transducer elements 1702 on the die 1612, digital data outputs from the RX circuits may be fed to a multiplexer (MUX) 1724 and then to a multiplexed digital processing block 1726 in the signal conditioning/processing circuit 1610. In other embodiments where the number of transducer elements 1702 is larger than the number of RX circuits (e.g., several transducer elements 1702 provide signals to a single RX circuit), the digital data from the RX circuits may be fed to the MUX 1724, which multiplexes the digital data and outputs multiplexed data to the multiplexed digital processing block 1726. Processed data may be output from the multiplexed digital processing block 1726 (and output from the semiconductor die 1612) via, e.g., the one or more high-speed serial output ports 1614 for, e.g., producing an image of the target. As will be appreciated, the MUX 1724 is optional; parallel signal processing may be performed in some embodiments of the present technology. Alternative implementations of the signal conditioning/processing circuit 1610 are described below.

### Probe Configurations

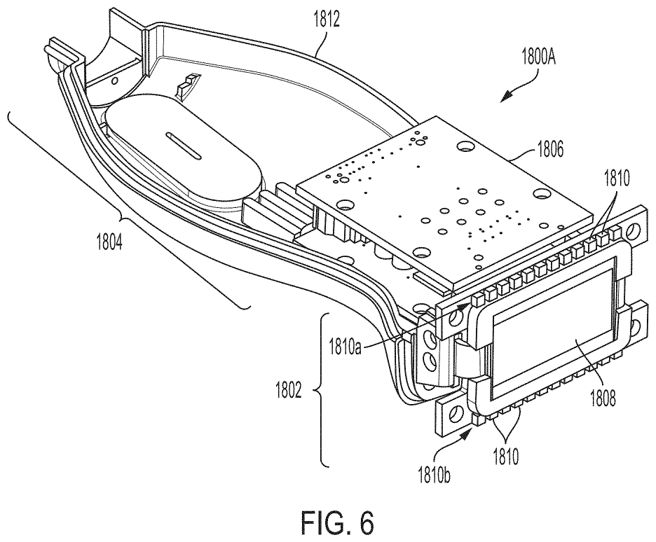

FIGS. 6, 7, and 8 schematically show different configurations of a photoacoustic imaging probe according to embodiments of the technology described herein. Each of the configurations is structured to enable the probe to be readily grasped by an operator, using a single hand of the operator. In some embodiments, the probe may be portable and may include a power source (e.g., a battery) and circuitry for wireless communication with, for example, an external computer or workstation. In some embodiments, the probe may have cabling that enables transfer of power and/or data.

FIG. 6 shows a cut-away illustration of a photoacoustic imaging probe 1800A according to some embodiments of the present technology. The probe 1800A may include a head portion 1802 and a handle portion 1804. Part of the handle portion 1804 is cut away to illustrate its interior. The head portion 1802 may include one or more array(s) of optical emitters 1810 (e.g., the diode array(s) 1603) and an transducer device 1808.

As discussed above, the array(s) of optical emitters 1810 may comprise LEDs, or laser diodes, or a combination of LEDs and laser diodes. Also as discussed above, the array(s) of optical emitters 1810 may comprise diodes of different wavelengths for irradiating the target to cause the target to produce ultrasonic waves according to the photoacoustic effect.

Although not shown in FIG. 6, the head portion 1802 may optionally comprise one or more Q-switched laser(s).

As discussed above, the transducer device 1808 may comprise one or more array(s) of ultrasonic transducers (e.g., the transducer array(s) 1602). Also as discussed above, the ultrasonic transducers may be MEMS devices, and may be ultrasound-a-chip devices having electronic circuitry integrated on the same semiconductor chip and the MEMS devices.

The handle portion 1804 may be sized to fit in an operator's hand, and may be comprised of a housing 1812 or a portion of the housing 1812. As shown in FIG. 5, the housing 1812 may have a hollow interior in which various components of the photoacoustic imaging probe 1800A may be accommodated. For example, the housing 1812 may hold therein a battery and/or a printer circuit board (PCB) containing off-chip electronics operatively connected to the transducer device 1808 and the array(s) of optical emitters 1810. For instance, the PCB may include a driver and/or a controller for providing an operator interface for a display of the photoacoustic imaging probe 1800A. The housing 1812 also may accommodate therein or thereon structural members for holding the transducer device 1808 and the array(s) of optical emitters 1810 in place.

The head portion 1802 may be structured such that the transducer device 1808 and the array(s) of optical emitters 1810 are aligned to face the target during imaging, so that a region of the target being irradiated by light from the optical emitters 1810 and directly facing the optical emitters 1810 also directly faces the receiving surfaces of the transducer device 1808, so that the photoacoustically generated ultrasonic waves may be captured efficiently. That is, the transducer device 1808 may be centrally located (or nearly centrally located) with respect to the array(s) of optical emitters 1810 to ensure efficient capturing of the ultrasonic waves generated by the target.

As shown in FIG. 6, the array(s) of optical emitters 1810 may comprise first and second arrays 1810a and 1810b located at a periphery of the transducer device on opposite sides of the transducer device 1808, such that the transducer device 1808 is positioned between the first and second arrays 1810a and 1810b. As will be appreciated, although the first and second arrays 1810a and 1810b are each shown to include a single row of the optical emitters 1810, they are not so limited and may each include multiple rows of the optical emitters 1810.

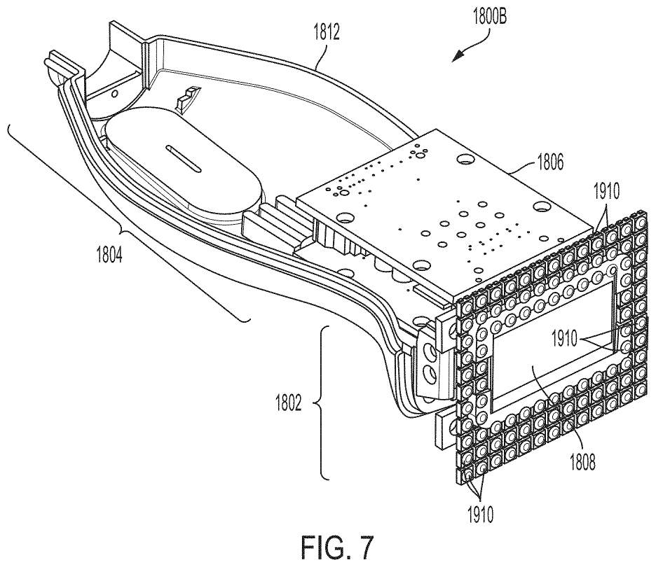

FIG. 7 shows a cut-away illustration of a photoacoustic imaging probe 1800B according to an embodiment of the present technology. Features of FIG. 7 that may be the same as of analogous to corresponding features in FIG. 6 may have the same reference numerals and will not be described again. The head portion 1802 of the probe 1800B may include one or more array(s) of optical emitters 1910 surrounding each of four peripheral sides (two short side and two long sides) of the transducer device 1808. The number of rows of the optical emitters 1910 on each of the peripheral sides may be the same or may be different. In the illustrated example, two rows of the optical emitters are arranged adjacent each of the short sides, and three rows of the optical emitters are arranged adjacent each of the long sides. The area of the array(s) of the optical emitters 1910 may be, for example, in the range of about 30 mm to 50 mm by about 30 mm to 50 mm, which may enable imaging of surface and near surface regions of the target.

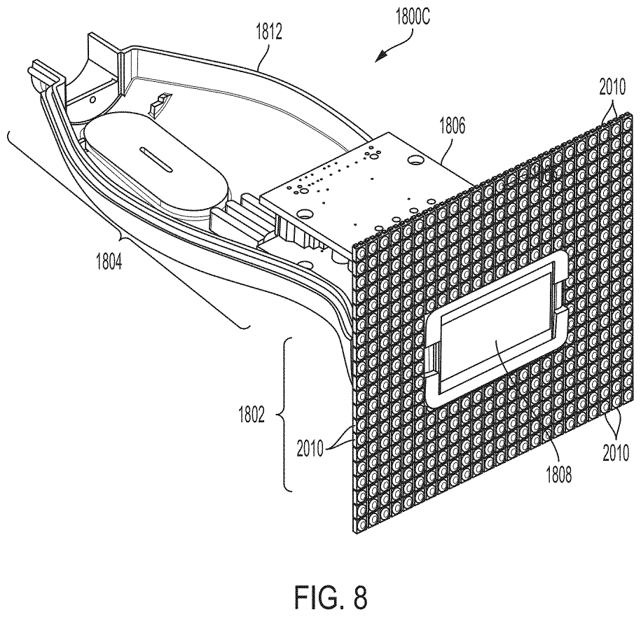

As will be appreciated, for imaging deeper into the target, a larger number of diodes may be used. FIG. 8 shows a cut-away illustration of a photoacoustic imaging probe 1800C according to an embodiment of the present technology. Features of FIG. 8 that may be the same as of analogous to corresponding features in FIG. 6 may have the same reference numerals and will not be described again. The head portion 1802 of the probe 1800C may include one or more array(s) of optical emitters 2010 surrounding each of four peripheral sides of the transducer device 1808. In the illustrated example, several rows of the optical emitters 2010 are arranged adjacent to each of the peripheral sides. For deep imaging of the target, the optical emitters 2010 may be arranged in one or more array(s) that are arranged in an area of about 70 mm to 100 mm wide on each side may be used. For example, a square array of the optical emitters 2010 having a width of 80 mm on each side may be used. Imaging deeper regions of the target is possible by increasing the lateral dimensions of the light-emission area of the optical emitters 2010, because the greater intensity of the emitted light ensures that, even with the amount of light diluted at the target by lateral expansion due to scattering, and even with the amount of light attenuated due to absorption-related attenuation with depth, a sufficient intensity of light reaches deeper into the target to cause photoacoustic generation of ultrasonic waves from the deeper regions to occur. Such deeper imaging may, in particular, be useful for imaging breast tissue.

In some embodiments of the present technology, the target may be irradiated or illuminated on multiple different sides to cause photoacoustic generation of ultrasonic waves in an entire thickness of the target. This may enable a three-dimensional image to obtained for an entire depth of the target. For example, by illuminating a breast target with optical emitters on opposite sides (e.g., left and right sides, or top and bottom sides), photoacoustic imaging may be performed for an entire depth of the breast. Two or more photoacoustic imaging probes (e.g., 1800C) may be used, or a single such probe may be used and additional illumination may be achieved by extending the emission area of the optical emitters (e.g., 2010) (e.g., by using multiple panels of LEDs) and/or by using a flexible panel of diodes. Signal Processing Considerations and Configurations

As noted above, diodes typically can generate a few W of peak optical power, whereas a Q-switched laser conventionally used for photoacoustic imaging typically can generate optical power in the MW range. Conventionally, as discussed above, a high-power Q-switched laser is operated to emit one optical pulse, which effectively illuminates an entire volume of the target, and to detect a pulse of photoacoustic waves generated by the target, which many have MPa of acoustic pressure, depending on absorption by one or more material(s) (e.g., tissue(s)) of the target. The time for the acoustic pulse to reach the detector (e.g., the transducer array(s) discussed above), at the speed of sound, is used to determine the depth in the target at which the optical pulse was absorbed. Lateral imaging of the target may be achieved by scanning the detector laterally, either by physical movement of a fixed-focus detector, or by use of a phased array imager.

In contrast to the conventional approach, diodes can only generate a few Pa of acoustic pressure at the target, which may yield an even lower pressure when propagated to and expanded at the detector. Sensing of such a lower pressure may require the diodes to emit longer pulse trains of light to irradiate the target, with a repetition rate of, e.g., a few MHz, to be averaged over tens of ms to reduce noise, as discussed above. The pulse trains can have an even lower pulse rate (e.g., in the 100 kHz range), to achieve a lower attenuation of the acoustic pulse, such as when probing deep tissue of the target or when going through bone (e.g., through the skull for brain imaging).

As noted above, the longer pulses permitted by user of a lower-power diode, in comparison with a Q-switched laser, may be used to probe deeper into the target. For depth determination considerations, one approach may be to use a short pulse train of one to five pulses at a few MHz (e.g., 1 MHz to 5 MHz). For instance, a pulse train of 5 pulses at 5 MHz may have a time duration of 1 μs, during which time the pulse train will travel 1.5 mm, assuming a typical sound speed of 1.5 km/s. By waiting until the farthest element (e.g., deepest tissue) of measurement to return an acoustic signal to the ultrasonic transducers before sending any more pulses, any interference between return signals originating at different depths may be eliminated. For example, to probe 50 mm deep into the target, the time for an acoustic signal (generated at all depths simultaneously by the optical pulse) to return to the ultrasonic transducers may be determined by (50 mm)/(1.5 km/s)=33 μs. If comparison cycles are performed to, e.g., to probe with ultrasonic waves launched at the target (which entails double the time for a round trip) and/or to probe with optical pulses of other colors (i.e., other wavelengths), the total cycle time for a full measurement may be more than 100 μs. As such, for real-time measurements, averaging the signal may be limited to a 10 Hz or 100 ms rate. In this example, averaging may be performed for 1000 returns at 100 microseconds per full cycle and 100 ms response time.

In order to get around the limited averaging, chirped or frequency-swept optical pulses and a heterodyne sensing algorithm may be used. Pulses at different times and originating from different depths may be allowed to overlap in at the ultrasonic transducers, with a heterodyne signal used to separate the different depths of the return signal at a given time, depending on the frequency of the pulses. As discussed herein, signal processing may be performed on-chip using ultrasonic transducers that are part of an ultrasound-on-a-chip device. The use of longer chirped or frequency-swept pulse trains and averaging may be implemented with any combination of: software, firmware, and hardware.

In one implementation, two-cycle optical bursts may be transmitted to the target, and the resulting acoustic signal may be received by an array of ultrasonic transducers and processed with a beamformer that has a double advancement speed (due to the one-way acoustic travel time in photoacoustic imaging). Otherwise, beamforming may be performed as in two-way ultrasonic imaging. Stated differently, the beamformer need not consider a time of flight for the transmitted optical signal (e.g., the optical bursts), but may consider only a time of flight of the received acoustic signal.

In another implementation, a chirped optical waveform spanning a set of frequencies may be transmitted, and the resulting acoustic signal received by the ultrasonic transducers may be processed by different methods. One method may be to capture, in its entirety, data across elements of a 1D or 2D array of the transducers and then processing a time dimension of the data with a cross-correlation chirp, for pulse compression. Cross-correlation may be performed in the time domain with a convolution, or in the frequency domain with a multiplication. Another method may be to process data on-the-fly by demodulating via multiplication with a ramped local oscillator that extends longer than the maximum frequency of the transmitted chirp. This may encode the depth with single frequency, such that a Fourier transformation may be used to convert the signal back to a photoacoustic image that is a depth map. Beamforming may be performed in these methods after pulse compression, to achieve a spatial image.

In another approach, processing of the photoacoustic waves emitted by the target (in response to exposure to light irradiation) and detected by a photoacoustic imaging probe (e.g., the probe 1800A) may be adapted from techniques used to process radar. (See, e.g., http://www.altimetry.info/radar-altimetry-tutorial/how-altimetry-works/from-radar-pulse-to-altimetry-measurements/5-1-2-2-full-deramp-technique/.) For example, when diodes of the probe emit a modulated chirp pulse (having a duration T, a frequency f, and a frequency-bandwidth B) towards the target, a “return” time at which photoacoustic waves from the target are detected may include a delay component corresponding to the estimated amount of time for the photoacoustic waves to reach the probe from the target, and another delay component that may be slightly shifted in frequency (Δf). Through processing that may involve mixing a deramped chirped pulse with a photoacoustic signal detected from the target, Fourier transformation processing may enable the frequency shift to be estimated, which in turn may enable a time delay (Δt) of the frequency shift to be estimated.

To produce a chirped pulse, a short optical pulse may be spread out or dispersed over time, such as via a dispersive delay line. The chirped pulse may be a frequency-modulated chirped pulse. When the photoacoustic waves are received by the probe, the corresponding photoacoustic signal may undergo pulse compression of (e.g., via an inverse matched filter), which compresses the received photoacoustic signal to a short pulse. The time delay (Δt) may be estimated to be inversely proportional to the bandwidth B according to the following:

Δt=(T/B)Δf.

With photoacoustic signals resulting from frequency-modulated optical pulses transmitted to the target (e.g., chirped optical pulses), photoacoustic signals may comprise discrete chirps, each generated from a different surface or material of the target and therefore each having slightly different delay times. Deramping of photoacoustic signals may involve mixing the photoacoustic signals with a copy of the transmitted chirped optical pulses, with a slightly shifted frequency. Such mixing may generate signals having this frequency difference (i.e., Δf), which in turn may enable the time delay (i.e., Δt) to be determined.

As will be appreciated, a frequency shift (or a time delay) from a region of the target may indicate that the photoacoustically generated waves originated from a different material than material in a nearby region, or may indicate that the photoacoustically generated waves originated from the same material but from different depths in the target. Thus, use of light of different wavelengths to irradiate the target may be beneficial for determining whether an observed frequency shift (or a time delay) is due to a different material, which may have different absorption characteristics for different wavelengths of light, or due to the same material at different depths, which likely would produce the same (or a very similar) observed frequency shift (or a time delay). Photoacoustic imaging may be used advantageously to detect the presence of an abnormal growth (e.g., a tumor) in the target, but determining the presence of unusual tissue material in, e.g., an organ of the target.

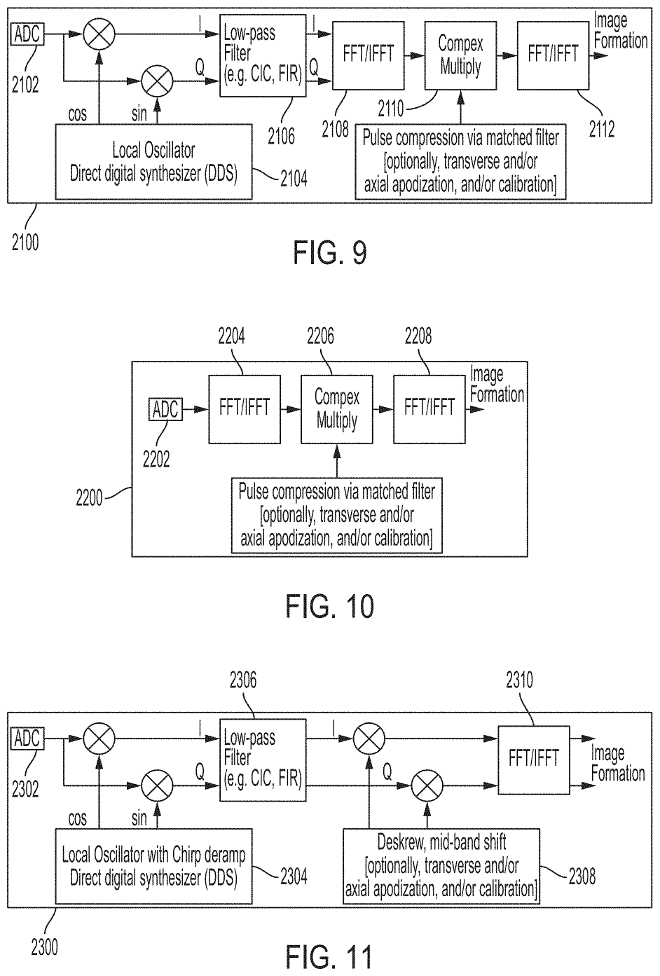

FIG. 9 shows a block diagram for a configuration of a signal processor 2100 that may be used to process signals output from ultrasonic transducers (e.g., the transducer device 1808) of a photoacoustic imaging probe (e.g., the probe 1800A), according to some embodiments of the technology presented herein. For example, the signal processor 2100 may comprise at least a part of the signal conditioning/processing circuit 1610.

The signal processor 2100 may receive a digitized signal from the transducers or an analog signal that may be digitized by an ADC 2102. The signal processor 2100 also may receive a copy of the optical signal (e.g., chirped pulses of light) emitted to the target, which may be digitized by the ADC 2102. The digitized signals may undergo deramp processing and other processing to obtain deramped signals having a cosine component I and a sine component Q. For example, a local oscillator 2104, which may include a direct digital synthesizer (DDS), may be used for such processing. The I and Q components then may undergo low-pass filtering at a low-pass filter 2106. For example, the low-pass filter 2106 may be a finite impulse response (FIR) filter, such as a cascaded integrator-comb (CIC) filter. After low-pass filtering, the filtered I and Q components may be provided to a Fourier transformation unit, at which the filtered I and Q components undergo a Fast Fourier Transformation (FFT) routine 2108. For example, the FFT routine 2108 may be a one-dimensional FFT routine. The Fourier transformation unit may comprise one or more executable routines stored in a memory (not shown) of the signal processor 2100, and may be executed by a microprocessor (not shown) of the signal processor 2100. Signals resulting from the FFT routine 2108 may be provided to a complex compression routine 2110, at which the signals undergo pulse compression via a matched filter. As discussed above, the pulse compression may compare data obtained from the chirped pulses of light with data obtained from the photoacoustic signals to determine frequency shifts (or timing delays), to obtain depth information and/or tissue-difference information. Optionally, the complex compression routine may include transverse and/or axial apodization of the signal, and/or calibration of the signal. A signal resulting from the complex compression routine 2110 may undergo an inverse FFT routine 2112 at the Fourier transformation unit, with the resulting data outputted for image formation. For example, the resulting data may be used to generate a planar or two-dimensional image in the time domain or the frequency domain, which (if applicable) may be transformed to a three-dimensional image by adding depth information to the two-dimensional image.

FIG. 10 shows a block diagram for a configuration of a signal processor 2200 that may be used to process signals output from ultrasonic transducers (e.g., the transducer device 1808) of a photoacoustic imaging probe (e.g., the probe 1800A), according to some embodiments of the technology presented herein. For example, the signal processor 2200 may comprise at least a part of the signal conditioning/processing circuit 1610. The signal processor 2200 may be, in some respects, a variation of the signal processor 2100.

The signal processor 2200 may receive a digitized signal from the transducers or an analog signal that may be digitized by an ADC 2202. The signal processor 2200 also may receive a copy of the optical signal (e.g., chirped pulses of light) emitted to the target, which may be digitized by the ADC 2202. The digitized signals may be provided to a Fourier transformation unit at which the digitized signals may undergo a deramp processing and a Fast Fourier Transformation (FFT) routine 2204. For example, the FFT routine 2204 may be a one-dimensional FFT routine. The Fourier transformation unit may comprise one or more executable routines stored in a memory (not shown) of the signal processor 2200, and may be executed by a microprocessor (not shown) of the signal processor 2200. Signals resulting from the FFT routine 2204 may be provided to a complex compression routine 2206, at which the signals undergo pulse compression via a matched filter. As discussed above, the pulse compression may compare data obtained from the chirped pulses of light with data obtained from the photoacoustic signals to determine frequency shifts (or timing delays), to obtain depth information and/or tissue-difference information. Optionally, the complex compression routine may include transverse and/or axial apodization of the signal, and/or calibration of the signal. A signal resulting from the complex compression routine 2206 may undergo an inverse FFT routine 2208 at the Fourier transformation unit, with the resulting data outputted for image formation. For example, the resulting data may be used to generate a planar or two-dimensional image in the time domain or the frequency domain, which (if applicable) may be transformed to a three-dimensional image by adding depth information to the two-dimensional image.

FIG. 11 shows a block diagram for a configuration of a signal processor 2300 that may be used to process signals output from ultrasonic transducers (e.g., the transducer device 1808) of a photoacoustic imaging probe (e.g., the probe 1800A), according to some embodiments of the technology presented herein. For example, the signal processor 2300 may comprise at least a part of the signal conditioning/processing circuit 1610.

The signal processor 2300 may receive a digitized signal from the transducers or an analog signal that may be digitized by an ADC 2302. The signal processor 2300 also may receive a copy of the optical signal (e.g., chirped pulses of light) emitted to the target, which may be digitized by the ADC 2302. The digitized signals may undergo deramp processing and other processing to obtain deramped signals having a cosine component I and a sine component Q. After deramping, the I and Q components may undergo low-pass filtering at a low-pass filter 2306. For example, the low-pass filter 2106 may be a finite impulse response (FIR) filter, such as a cascaded integrator-comb (CIC) filter. After low-pass filtering, the filtered I and Q components may undergo a routine 2308 for deskewing and mid-band shifting before being provided to a Fourier transformation unit, at which the deskewed I and Q components undergo a Fast Fourier Transformation (FFT) routine 2310 to obtain frequency-difference information (e.g., Δf) and phase-difference information. Optionally, the routine 2308 may include transverse and/or axial apodization of the I and Q components, and/or calibration of the I and Q components. Data output from the FFT routine 2310 may be used for image formation.

Any use of the terms “program,” “application,” or “software” herein may refer, in a generic sense, to any type of computer code or set of processor-executable instructions that may be employed to program a computer or other processor to implement various aspects of embodiments as discussed above. Additionally, according to one aspect, one or more computer programs that when executed perform methods of the disclosed technology provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Further, some actions are described as taken by an “operator” or a “user.” It should be appreciated that these terms need not refer to a single individual, and that in some embodiments, actions attributable to an “operator” or a “user” may be performed by a team of individuals and/or an individual in combination with computer-assisted tools or other mechanisms. Further, it should be appreciated that, in some instances, a “target” being imaged may be the same person as an “operator.” For example, an individual may be imaging himself or herself with a photoacoustic probe and, consequently, may act as both the “target” being imaged and the “operator” of the probe.

Any use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The indefinite articles “a” and “an,” as used herein in the specification and in the claims, unless clearly indicated to the contrary, should be understood to mean “at least one.”

Any use of the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Any use of the phrase “equal” or “the same” in reference to two values (e.g., distances, widths, etc.) means that two values are the same within manufacturing tolerances. Thus, two values being equal, or the same, may mean that the two values are different from one another by ±5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “substantially” if used herein may be construed to mean within 95% of a target value in some embodiments, within 98% of a target value in some embodiments, within 99% of a target value in some embodiments, and within 99.5% of a target value in some embodiments. In some embodiments, the term “substantially” may equal 100% of the target value.

Also, some of the embodiments described above may be implemented as one or more method(s), of which some examples have been provided. The acts performed as part of the method(s) may be ordered in any suitable way. Accordingly, embodiments may be constructed in which acts are performed in an order different than illustrated or described herein, which may include performing some acts simultaneously, even though shown as sequential acts in illustrative embodiments.

Further, although advantages of the present invention may be indicated, it should be appreciated that not every embodiment of the invention will include every described advantage. Some embodiments may not implement any features described as advantageous herein. Accordingly, the foregoing description and attached drawings are by way of example only.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

What is claimed is:

1. A photoacoustic imaging apparatus, comprising:
an ultrasound-on-a-chip device attached to a housing, the ultrasound-on-a-chip device comprising a plurality of ultrasonic transducers;
an optical emitter attached to the housing, the optical emitter comprising an array of diodes arranged at a periphery of the plurality of ultrasonic transducers; and
a controller enclosed at least partially in the housing and configured to control the optical emitter to emit pulses of light, to control the plurality of ultrasonic transducers to detect and measure ultrasonic waves emitted from a target to be imaged in response to exposure to the pulses of light, and to convert the ultrasonic waves to digital signals.

2. The photoacoustic imaging apparatus of claim 1, wherein
the ultrasound-on-a-chip device comprises the controller, and
the controller is configured to process the digital signals to produce image-formation data.

3. The photoacoustic imaging apparatus of claim 2, wherein the controller is configured to perform parallel processing of the digital signals from the plurality of ultrasonic transducers.

4. The photoacoustic imaging apparatus of claim 1, wherein the array of diodes comprises a plurality of light-emitting diodes (LEDs), or a plurality of laser diodes, or a combination of at least one LED and at least one laser diode.

5. The photoacoustic imaging apparatus of claim 4,
wherein the plurality of LEDs comprise at least two LEDs that emit light having different wavelengths from each other, and
wherein the controller is configured to control the optical emitter to perform tunable imaging of the target using the different wavelengths.

6. The photoacoustic imaging apparatus of claim 5, wherein the controller is configured to control the optical emitter to emit light selectively according to a desired wavelength for imaging the target.

7. The photoacoustic imaging apparatus of claim 1, wherein the optical emitter comprises at least one Q-switched laser.

8. The photoacoustic imaging apparatus of claim 1, wherein the array of diodes is arranged to surround a periphery of the plurality of ultrasonic transducers.

9. The photoacoustic imaging apparatus of claim 1, further comprising:
a handle sized to be held by a single hand of a user, the handle forming at least a portion of the housing; and
a battery held within the housing and configured to provide electric power to the ultrasonic transducers, the optical emitter, and the controller.

10. The photoacoustic imaging apparatus of claim 1, wherein the plurality of ultrasonic transducers comprise any one or any combination of:
at least one capacitive micromachined ultrasonic transducer (CMUT), and
at least one piezoelectric micromachined ultrasonic transducer (PMUT).

11. The photoacoustic imaging apparatus of claim 10, wherein the plurality of ultrasonic transducers operate in a range of approximately 1 MHz to approximately 40 MHz.

12. The photoacoustic imaging apparatus of claim 10, wherein the plurality of ultrasonic transducers operate in a range of approximately 40 MHz to approximately 180 MHz.

13. The photoacoustic imaging apparatus of claim 10, wherein
the plurality of ultrasonic transducers comprise a plurality of MEMS transducers configured to detect ultrasonic waves emitted from a volume of material in the target, and
the controller is configured to process the ultrasonic waves emitted from the volume of material to produce a 3D image of the target.

14. The photoacoustic imaging apparatus of claim 1, wherein the controller is configured to perform chirp processing on pulse trains and to control the array of diodes to perform emission of chirped pulses.

15. A photoacoustic imaging system, comprising:
a photoacoustic imaging apparatus comprising:
a plurality of ultrasonic transducers supported by a housing, and
an optical emitter supported by the housing, the optical emitter comprising an array of diodes arranged at a periphery of the plurality of ultrasonic transducers;
a controller configured to control the optical emitter to emit pulses of light and the ultrasonic transducers to detect and measure ultrasonic waves emitted from a target to be imaged in response to exposure to the pulses of light, and to convert the ultrasonic waves to digital signals; and
a signal processor configured to determine depth information from the digital signals and to output image data for a three-dimensional image of the target.

16. The photoacoustic imaging system of claim 15, wherein the controller is configured to perform chirp processing on pulse trains of light and to control the optical emitter to perform emission of chirped pulses.

17. The photoacoustic imaging system of claim 15, wherein the signal processor is configured perform any one or any combination of:
analog filter,
digital filtering,
deramp processing,
cross-correlation processing,
pulse-compression processing, and
heterodyne processing to determine the depth information.

18. A photoacoustic imaging method, comprising:
scanning a target using a photoacoustic imaging probe comprising:
an ultrasound-on-a-chip device that includes a plurality of ultrasonic transducers and control circuitry supported by a housing, and
an optical emitter supported by the housing, the optical emitter comprising an array of diodes arranged at a periphery of the plurality of ultrasonic transducers; and
performing, using the control circuitry, signal processing on ultrasonic waves photoacoustically generated by target to convert the ultrasonic waves to digital signals used to produce a photoacoustic image,
wherein the scanning comprises:
irradiating the target with light from the array of diodes, and
detecting the ultrasonic waves at the plurality of ultrasonic transducers.

19. The photoacoustic imaging method of claim 18, wherein the irradiating of the target comprises irradiating the target with chirped pulses of light from the array of diodes.

20. The photoacoustic imaging method of claim 18, further comprising:
performing an background scan of the target by emitting ultrasonic waves from the plurality of ultrasonic transducers and receiving back-scattered ultrasonic waves from the target;
processing the back-scattered ultrasonic waves from the target to produce an ultrasound image; and
comparing the ultrasound image with the photoacoustic image to determine features of the target that appear in both the ultrasound image and the photoacoustic image, and features of the target that appear in one but not both of the ultrasound image and the photoacoustic image.
